(19) United States
(12) Patent Application Publication
(10) Pub. No.: US 2008/0060662 A1
(43) Pub. Date: Mar. 13, 2008

(54) Protected Information Management Device and Method

(75) Inventors: Joon Oh, Moraga, CA (US); Mark L. Marchan, Cordova, TN (US)

Correspondence Address: HAYNES AND BOONE, LLP, 901 Main Street, Suite 3100, Dallas, TX, 75202, US

(73) Assignee: WARSAW ORTHOPEDIC INC., Warsaw, IN (US)

(21) Appl. No.: 11/462,246

(22) Filed: Aug. 3, 2006

(51) Int. Cl. A61B 19/00

(52) U.S. Cl. 128/897

(57) ABSTRACT

Embodiments of the invention include devices and methods for collecting clinical information about the performance of a medical device, and controlling the transmission of at least portions of the information. The information controlled may be protected health information or other personal or confidential information which may be controlled in accordance with PIPEDA, HIPAA, or other laws, regulations, or standards.

### FIELD OF THE INVENTION

The present invention relates generally to the field of managing private and non-private information, and more particularly relates to restricting access to private information such as protected health information (PHI), while making available associated information that may be useful in evaluating medical treatment.

### BACKGROUND

The Health Insurance Portability and Accountability Act (HIPAA) was passed by the U.S. Congress in 1996 and was signed into law. HIPAA addresses a number of needs perceived to exist within the collective healthcare systems of the United States. HIPAA took effect on Apr. 14, 2003. One provision under HIPAA relates to privacy of patient information. The HIPAA privacy provisions ensure that personal medical information shared with doctors, hospitals, and others who provide or pay for healthcare is protected from unauthorized disclosure.

HIPAA affects individuals and businesses that have access to patient records by imposing restrictions on how the individuals and businesses use and protect information. When a patient gives personal health information to an entity covered by the law, that information becomes protected health information (PHI). PHI includes any information about a person's physical or mental health, services rendered, or payment for the services. PHI also includes personal information connecting the patient to the records. PHI may be oral, audibly recorded, written, or in electronic form. Examples of information that connect personal health information to an individual patient include the patient's name, address, social security or other identification number, physicians' notes regarding the patient, and billing information.

As of Jan. 1, 2004, all Canadian businesses are required to comply with the privacy principles set out in a Canadian law entitled the Personal Information Protection and Electronic Documents Act (PIPEDA). The law protects personal information accessible to private sector organizations and provides guidelines for the collection, use, and disclosure of that information in the course of commercial activity. PIPEDA covers both traditional, paper-based businesses and on-line businesses. PIPEDA defines personal information as, “information about an identifiable individual,” and sensitive personal information, such as information which may include health or medical history, racial or ethnic origin, political opinions, religious beliefs, trade union membership, financial information, and sexual preferences. Personal information and sensitive personal information will also be referred to as PHI herein.

It is often necessary during the development and evaluation of medical devices to monitor the long-term efficacy of the medical devices. Therefore, it is necessary to associate particular medical devices with particular patients to accurately monitor performance of the devices. However, because of HIPAA and PIPEDA privacy rules, patients may not be identified by PHI to individuals or businesses not specifically authorized or equipped to receive and protect such information. Consequently, it is often necessary to “de-identify” device performance information from PHI, and then to protect codes that correlate the PHI and non-PHI associated with device performance.

A number of systems currently exist that are useful in collecting information, such as device performance information, from patients at a health care providers' site. These systems collect PHI and non-PHI, and then transmit all of the information to a computer where the information will be de-identified. A significant disadvantage of such systems is that the PHI must be transmitted away from the health care provider to be processed. If de-identification and other data processing were to take place at the health care providers' sites, more significant computer processing resources would have to be stationed with each health care provider. Additionally, such a system may not provide a means for the health care provider to benefit from data collected by other health care providers. An improved system may collect information at the heath care provider's location, de-identify PHI from the record, and then transmit only non-PHI to other parties for use in actions such as device performance analysis and clinical evaluation. In an improved system, non-PHI to be transmitted to the other parties may be associated with a designator linking the non-PHI to a particular patient. The linking designator's association with the PHI in an improved system may reside with the health care provider at all times, providing enhanced security for the information.

### SUMMARY

One embodiment of the invention is a computer system for collecting clinical information regarding degrees of success or failure resulting from implantation of a medical device. The system may include a local computing device on which PHI and non-PHI are stored. Embodiments of the local computing device including at least an authentication sequence, a tasking sequence, and a communications interface capable of communicating non-PHI over a network, but restricted from communicating PHI over the network. The system may also include a central computing device for receiving non-PHI from the local computing device and for processing non-PHI. In some embodiments, non-PHI is correlated with an identifier, and the identifier is associated with portions of PHI in the local computing device.

Another embodiment of the invention is a computer system for collecting clinical information including a local computing device and a central computing device. Embodiments of the local computing device include data entry pages and a local database capable of receiving data from the data entry pages. PHI and non-PHI may be stored in the local database, and embodiments of the local computing device are capable of communicating over a network, but restricted from communicating PHI over the network. The central computing device is for receiving non-PHI from the local computing device and for processing the non-PHI. The central computing device may include a web server connectable with the local computing device for receiving information over the network, and a database server for storing and processing non-PHI.

Yet another embodiment of the invention is a clinical evaluation system including a medical device for treating a medical condition and a local computing device into which information is input, the information comprising PHI, non-PHI, and medical implant performance information related to treatment of the medical condition. The information regarding the performance of the medical implant may include one or both of PHI and non-PHI. The system may also include a central computing device connectable to the local computing device through a network. Embodiments of the central computing device are enabled to receive non-PHI, but not able to receive PHI from the local computing device.

An embodiment of the invention is a local computing device with a memory device in which PHI and non-PHI are stored, and computer readable instructions providing a communications interface that enables the local computing device to transmit non-PHI over a network to another computing device, but restricts the local computing device from communicating PHI over the network. In some embodiments, the local computing device is a portable device retained within the control of a health care provider.

Still another embodiment of the invention is a method of evaluating medical outcomes resulting from implantation of a medical device. The method may include collecting PHI and non-PHI from a patient in which the medical device has or will be implanted and entering at least a portion of the PHI and the non-PHI into a local computing device. Further the method may include transmitting at least a portion of the non-PHI to a central computing device, preventing transmission of the PHI to the central computing device; and evaluating at least portions of the non-PHI transmitted to the central computing device.

An embodiment of the invention is a computer readable media containing instructions to enable collection of clinical information. The instructions may include instructions to display data entry pages into which PHI and non-PHI may be added, instructions to store PHI and non-PHI in a local database, instructions to communicate non-PHI over a network, and instructions restricting communication of PHI over the network.

### BRIEF DESCRIPTION OF THE DRAWINGS

FIG. 1 is a conceptual diagram for embodiments of the invention.

FIG. 2 is an operative block diagram for embodiments of the invention.

FIG. 3 is a representation of a computer screen presented to a user in some embodiments to assist with management of scheduled events during a month.

FIG. 4 is a representation of a computer screen presented to a user in some embodiments to assist with management of scheduled events during a week.

FIG. 5 is a representation of a computer screen presented to a user in some embodiments to assist with management of scheduled events during a day.

FIG. 6 is a flowchart directed to method embodiments of the invention.

### DETAILED DESCRIPTION

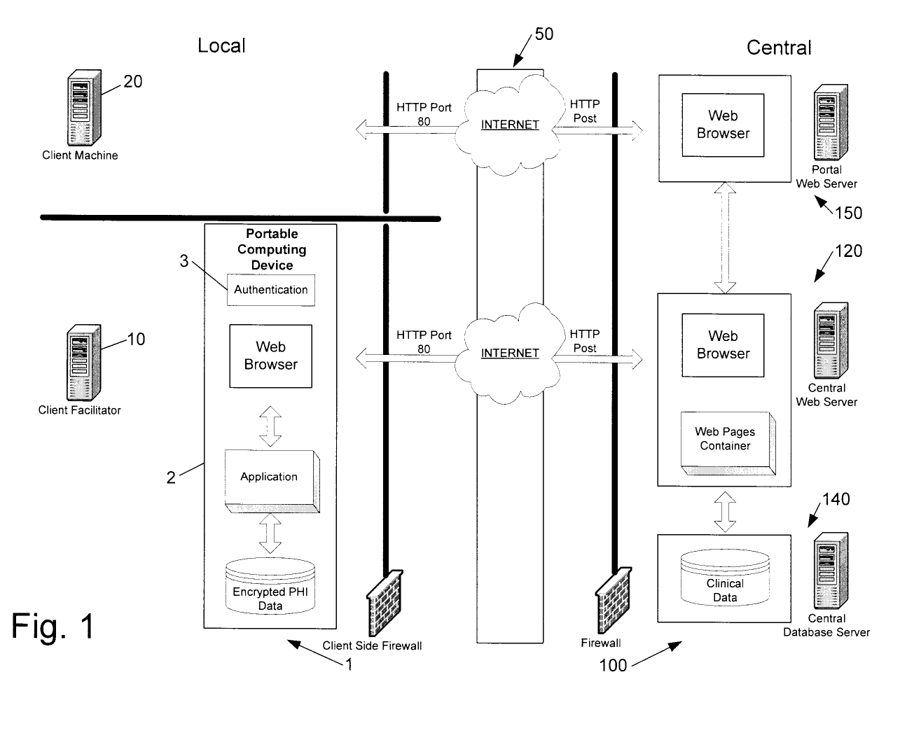

FIG. 1 illustrates a conceptual diagram of a computer system for collecting clinical information regarding degrees of success or failure resulting from implantation of a medical device in a patient. A local computing device 1 on which protected health information (PHI) and non-PHI may be stored is shown. The local computing device 1 may include one or more of a portable computing device 2, a client facilitator 10, and a client machine 20.

The term non-PHI as used herein may include PHI that has been de-identified; wherein PHI is de-identified when personal information or information which may be combined to identify a specific person is disassociated or removed.

The local computing device 1 as illustrated is connected to a central computing device 100 by a network 50. The central computing device 100 in some embodiments is for receiving non-PHI from the local computing device 1 and for processing non-PHI. The central computing device 100 may include one or more of a central web server 120, a central database server 140, and a portal web server 150. In some embodiments, non-PHI is correlated with an identifier, and the identifier is associated with portions of PHI in the local computing device 1.

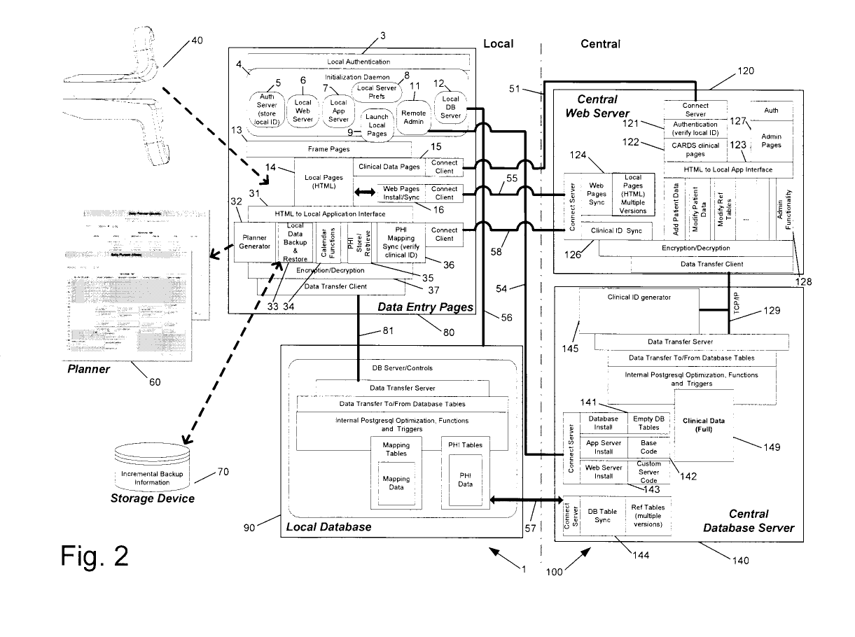

The local computing device 1 may include a portable computing device 2 that also includes a Universal Serial Bus (USB) device. The portable computing device 2 could also be a laptop computer, a handheld computing device, a memory card, a disc drive, a tape recording device, a “smart card,” a cellular telephone, or any other device capable of storing data. The local computing device 1 may be a stand-alone computing device, memory device, or a combination of memory and stand-alone computing devices. For example, the local computing device 1 illustrated in FIG. 1 may include one or more of the portable computing device 2, the client facilitator 10, and the client machine 20. In some embodiments, the portable computing device 2 is connected to the client facilitator 10, and the two devices in combination execute instructions to accomplish functions such as those detailed in association with FIG. 2. One or all of the portable computing device 2, the client facilitator 10, and the client machine 20 include at least a processor, a memory device, and a bus. The bus is for communicating information at least between the processor and the memory device.

The local computing device 1 may include a portable computing device 2 that includes a USB memory device and a processor combined into a single device. For example, the portable computing device 2 may include a “USB pocket server” as has been offered by Realm Systems. One version of the USB pocket server uses a 400 MHz PowerPC Processor and has 64 MB of RAM. The device is powered through a USB connection to a host computer to which it is connected. The USB pocket server requires no special software to be executed by the host computer and boots automatically. The USB pocket server can access the host computer's peripherals and network resources.

FIGS. 1 and 2 illustrate embodiments of a communications interface capable of communicating non-PHI over a network 50, but restricted from communicating PHI over the network 50. The communication interface may include one or more of connections to the network 50 and software or other mechanisms or coding to control the transmission of signals over the network 50. For example, the communications interface illustrated in FIG. 2 includes a communications link 51 coupled between local clinical data pages 15 and a central web server 120 of central computing device 100. The network 50 may include one or more of a local area network, a wide area network, the Internet, and any other interface over which digital data may be exchanged.

FIG. 2 illustrates a number of data transfer, connect, control, and encryption/decryption interfaces, both from client and server sides. These devices will not otherwise be designated and functionally described herein. Their functions are understood by one skilled in the art.

The central computing device 100 as shown may be one or more computers. As illustrated in FIG. 1, the central web server 120, the central database server 140, and the portal web server 150 are separate computers that are interconnected. Alternatively, two or more of the functions of the computer may be resident on a single device. By way of example, and without limitation, one or both of the computers may include a PENTIUM 4 processor by Intel Corporation, and more specifically may include dual XEON processors. Each system may include at least two to four gigabytes of RAM. The central web server 120 of some embodiments may include at least 70 gigabytes of storage capacity. The central database server 140 of some embodiments may include at least 120 gigabytes of storage capacity. A RAID data storage algorithm and associated hardware may also be employed. Some embodiments may include hot swap capabilities for various server components.

In some embodiments, the central web server 120 may be loaded with the following software: Red Hat, version 9; Apache HTTP Server, version 2.0.54; Apache Tomcat Server, version 5.5; and J2SE JDK 5.0, update 5. The central database server 140 may be loaded with Red Hat, version 9 and Postgresql, version 8.0. Other functionally equivalent or otherwise capable programs may be employed in various embodiments.

The local computing device 1 illustrated in FIGS. 1 and 2 includes an authentication sequence 3 capable of controlling access to functionality of the local computing device 1. The authentication sequence 3 is represented graphically in FIGS. 1 and 2. The authentication sequence 3 may be carried out by execution of software code, by circuitry fabricated into the local computing device 1, or by any other effective execution mechanism or sequence. The authentication sequence 3 may require one or more of a username, password, or biometric authentication information. A biometric scanner may include fingerprint identification, voice recognition, retinal identification, or identification of other characteristics unique to an individual or class of users. Some commercially available USB devices with integrated biometric fingerprint scanners, for example, are capable of recognizing and authenticating five different users and may or may not additionally require a password.

The local computing device 1 illustrated in FIG. 2 shows an initialization daemon 4 that runs during operation of the device for the purpose of handling periodic service requests that are received. The initialization daemon 4 forwards requests to other programs or processes as appropriate. Programs and processes that may be running in the illustrated embodiment include an authentication server 5, a local web server 6, a local application server 7, a local server preferences application 8, a local pages launch application 9, a remote administration module 11, and a local database server 12.

The authentication server 5 contains a program that further manages user access to the system. In some embodiments, the authentication server 5 determines if a user has already logged into the system on the local computing device 1. The authentication is based on a local identification code assigned to each user. Local identification code data may be stored in a predetermined location on a user partition of a hard drive, such as the hard drive of the client facilitator 10. If a local identification code data file does not exist in the predetermined location, the program may create a file on a local machine or network machine for current and future use. The local identification codes are used to determine the identification of the device that is making requests of the central web server 120. The identification code may be sent with all requests and stored with activity logs. In some embodiments, if the central web server 120 determines that an identification code is associated with a local computing device that has been reported lost, stolen, or inactivate, the central web server 120 will not honor any request associated with the local identification code.

The local web server 6 and local application server 7, alone or in combination, may contain programs for initiating presentation of web pages, such as local pages 14, to a user. The programs may also perform other processing and manage access to and receiving information from the network 50. In one embodiment, the local application server 7 is a Tomcat application server from the Apache Software Foundation. The program may execute Java servlets and render web pages that include Java Server Page (JSP) coding. The Tomcat application server may be used as both an HTTP server and a JSP server. In other embodiments, the Tomcat server, acting as the local application server 7, may perform solely as the JSP server, and an Apache HTTP server will be used as an HTTP server. In the latter configuration, the Apache HTTP server may be the local web server 6.

The local server preferences application 8 contains information regarding local user preferences regarding the form, presentation, and content of data entry pages 80. The local server preference information is associated with the local identification code for the user and local computing device 1 being operated.

The local pages launch application 9, as illustrated, contains a program that opens the local pages 14 and local clinical data pages 15. The local pages 14 are defined by a set of frame pages 13. The local pages 14 and local clinical data pages 15 illustrated as part of the local computing device 1 include at least one tasking sequence wherein interfaces for inputting and reading PHI and non-PHI are presented. In some embodiments, the computer code enabling the interfaces for inputting and reading PHI and non-PHI is stored on the local computing device 1 in hypertext markup language (HTML). More specifically, the code may be stored in HTML on a portable computing device 2 that is a USB device, and launched from a predefined shortcut on the USB device.

The local clinical data pages 15 communicate with the central web server 120 as noted above. The central web server 120 contains central clinical data pages 122 that exchange data with the local clinical data pages 15. A local identification authentication module 121 controls access to the central clinical data pages 122 by verifying the local identification code. In some embodiments, an application local to the web server 120 controls additions and modifications of patient data, reference data, and other central clinical data pages information through a central HTML to local application interface 123.

The central web server 120 may also enable administrative capabilities. As illustrated in FIG. 2, administrative pages 127 are accessible through an authentication process, and then are implemented through the central HTML to local application interface 123 in combination with administrative functionality programs 128.

As shown in FIG. 2, the central web server 120 exchanges data with the central database server 140 via a TCP/IP communications link 129. Both the clinical identification generator 145 data and clinical data from a clinical data storage module 149 may be transmitted over the TCP/IP communications link 129. The internal function of the database within the central database server 140 is evident to one skilled in the art as depicted in FIG. 2, and will not be further discussed. Other database and database access configurations are contemplated by embodiments of the invention as would be functionally sufficient.

The remote administration module 11 contains a program that enables maintenance and updating of the local computing device 1. In one example, a USB portable computing device 2 may be maintained in response to commands initiated through the USB device via buttons or controls generated by web pages that are part of the data entry pages 80. For example, if a user wanted to reformat a USB device, a button on the USB device physically or a button generated from code stored on the USB device could be activated to cause the remote administration module 11 to connect with the database server 140 via connection 54 and download a current version of software. As illustrated, the software is stored in separate modules: a database install module 141, an application server install module 142, and a web server install module 143. The storage and function of these modules may be combined or partially combined in other embodiments. These modules individually or in combination with one or more of the modules may be referred to generally as a maintenance module.

The local database server 12 of the illustrated embodiment contains a program that enables communication between the initialization daemon 4 and the local database 90 via local database connection 56. As a result, the data entry pages 80 have access to the data stored in the local database 90.

As depicted in FIG. 2, a web pages install and synchronize module 16 enables the tasking sequence, through the initialization sequence, to compare software code stored in the local computing device 1 with software code stored in the central computing device 100. The web pages install and synchronize module 16 is connected to a web pages synchronize module 124 through a synchronization connection 55. In some embodiments, the web pages synchronize module 124 includes multiple versions of web pages that may be used by the local computing device 1, thereby enabling the web pages synchronize module 124 to compare and provide requested and updated versions of the web pages to the local computing device 1. Therefore, the software code representing the web pages in the local computing device 1 may be compared to the software code representing the web pages in the central computing device 100. If the software code stored in the local computing device 1 is an older version than the software code stored in the central computing device 100, the local computing device software code may be automatically updated in some embodiments. Alternatively, a notice can be provided to the user, allowing the user to make a choice between updating the software code and continuing to operate with the previously installed software code.

FIG. 2 also illustrates a medical device 40 for treating a medical condition about which data is collected under embodiments of the invention. The device illustrated is a spinal arthroplasty device. However, in other embodiments, the medical device 40 may be a device for addressing any medical condition. By way of example and without limitation, the medical device may be another spinal or orthopedic device, a defibrillator, pacemaker, or other device for treating the cardiopulmonary system, a device for treating neurological conditions, a drug or other substance delivery device, or a monitoring device.

A local application or launch container software of embodiments of the invention includes logic that will accomplish one or more of fetching, decrypting, and modifying PHI data. PHI data under control of the launch container software may be displayed for a user and may be linked with clinical data centrally stored on the central computing device 100. The launch container software in the illustrated embodiment interacts with the local pages 14, the local database 90, the central web server 120, an incremental backup data storage device 70, and a daily planner 60. The launch container software may be a Tomcat, version 5.5.9, application server from the Apache Software Foundation. Code may be initiated from locally stored web pages such as the local pages 14.

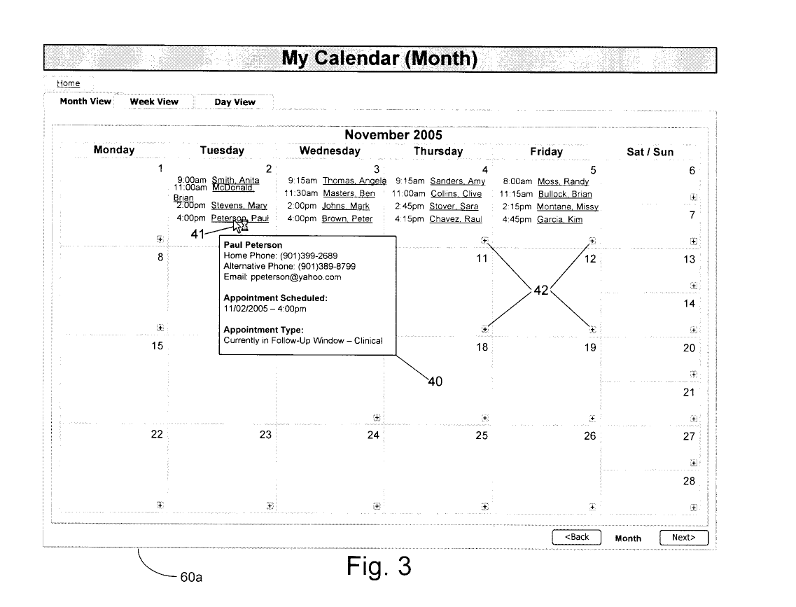

Referring to the graphical depiction of FIG. 2, a local HTML to local application interface 31 communicates with the local pages 14, and therefore, all of the components supplying data to the local pages 14. A planner generator 32 and calendar functions 34 interact to create a planner 60. The planner 60 displays actions to be carried out during the collection of clinical information regarding degrees of success or failure resulting from implantation of a medical device in a patient. Examples of actions that may be displayed include patient scheduling and compliance actions such as pain analyses questionnaire completion and other registration information collection, post-operative examinations and appointments, and additional procedures, as may be necessary. Examples of monthly, weekly, and daily planners 60a-60c are provided in FIGS. 3, 4, and 5 respectively. Other configurations for planners and similar displays or interfaces may be used to present and receive information. The planner generator 32 as described therefore uses PHI and non-PHI to calculate future patient compliance actions and other actions.

FIG. 3 illustrates a monthly planner 60a showing a number of patients' names and their associated appointment times on designated days. A detail box 40 is shown in the illustrated embodiment. The detail box 40 is initiated by pointing a cursor 41 at a particular patient name. A similar detail box may be associated with each patient name. The detail box 40 provides additional information about the patient with which the detail box 40 is associated. As shown, an appointment may be added by designating any plus key 42 associated with a day.

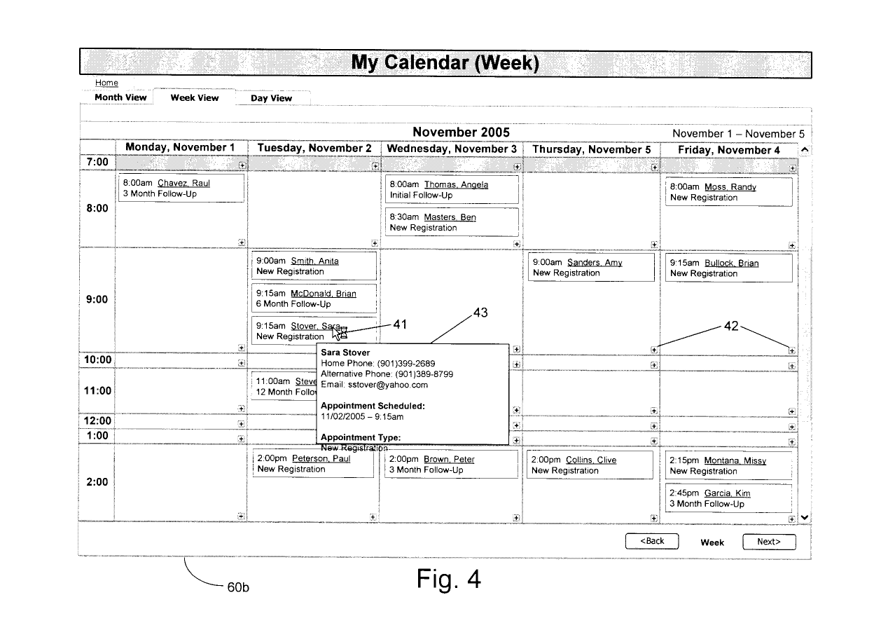

FIG. 4 shows a weekly planner 60b that list patients' names, appointment times, and a recorded reason for their appointment. Another detail box 43 is initiated by pointing the cursor 41 at a patient name.

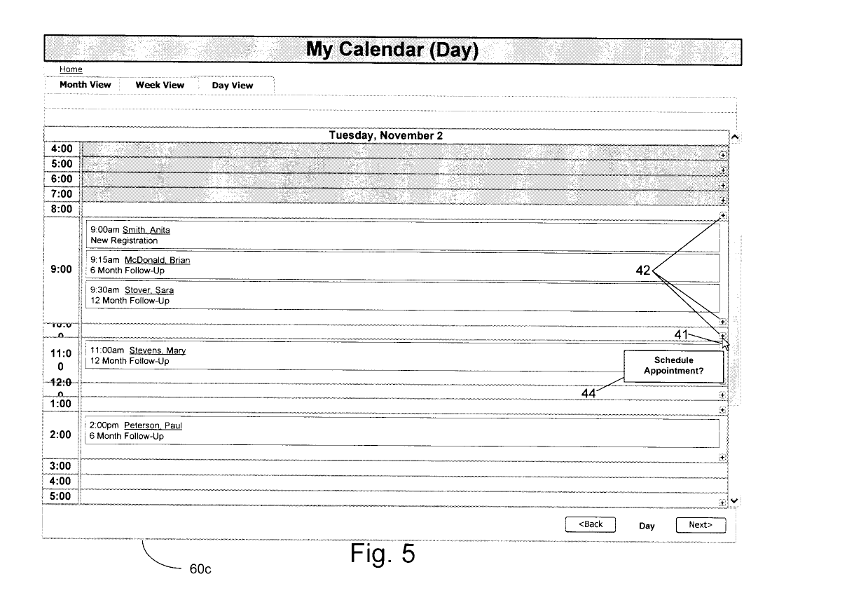

A daily planner 60c is illustrated in FIG. 5. A list of patient's names, appointment times, and a recorded reason for their appointment is shown within a time block that represents each appointment. Additional space is provided for notes or comments. Detail boxes may be associated with each name, and appointments may be added by designating any plus key 42, just as in association with the monthly and weekly planners. The cursor 41 is shown directed to the plus key 42. Rolling over the plus key may cause an information box 44 to appear that provides a user with the information that further designating the plus key 42 will enable new appointment information to be added.

As shown in FIG. 2, the local data backup and restore module 33 controls access to and storage of copies of data stored separate from a device such as a USB device when used as the local computing device 1. As discussed above, local identification code data may be stored in a predetermined location on a user partition of a hard drive, such as the hard drive of the client facilitator 10, or on a local network machine. Similarly, all data stored on a local device such as a USB device may be stored on another local machine for backup purposes. As depicted in FIG. 2, the machine on which local backup is accomplished is the incremental backup data storage device 70. In addition to the client facilitator 10 and a local network machine, backup may be accomplished on a secondary portable device such as a USB memory device, a laptop computer, a handheld computing device, a memory card, a disc drive, a tape recording device, a “smart card,” a cellular telephone, or any other device capable of storing data.

The PHI store and retrieve module 35 accomplishes data transfer tasks between the local pages 14 and the local database 90 with PHI data. Data transferred to and from the local database 90 may be encrypted by an encryption/decryption module 37 and is illustrated in FIG. 2. A local database connection 81 provides for data transfer between the data entry pages 80 and the local database 90. The internal function of the local database 90 is evident to one skilled in the art as depicted in FIG. 2, and will not be further discussed. Other database and database access configurations are contemplated by embodiments of the invention as would be functionally sufficient. Note that the reference table configuration for the local database 90, but not PHI data, is synchronized with the central database server 140 by interaction with a database table synchronize module 144, via table connection 57.

In some embodiments, the local computing device 1 and the central computing device 100 communicate regarding specific sets of data associated with particular devices and patients by assigning a unique identifier to each set of data. The unique identifier is referred to herein as a clinical identification code. The clinical identification codes are only correlated with PHI data within the local computing device 1. Only the clinical identification codes, non-PHI data, and data that is only PHI data when associated with other PHI data that is not being transmitted to the central computing device 100 are transmitted to the central computing device 100. This and other structures and methods of restricting the communication of PHI over the network 50 are contemplated by embodiments of the invention.

Because the clinical identification codes exist in both the local computing device 1 and the central computing device 100, it is necessary to synchronize between the devices periodically. This synchronization mechanism is depicted by a PHI mapping synchronize module 36 in the local computing device 1 and its connection to a clinical identification synchronize module 126 in the central computing device 100. Communication is via a clinical identification connection 58. A clinical identification generator 145 is part of the central database server 140. The clinical identification generator 145 supplies clinical identification codes for use by the central web server 120 and the data entry pages 80.

One function of embodiments of the central computing device 100 is to deliver non-PHI data to requesters. A requester may be a user with a portable computing device 2, such as a USB device. A requester may also be a user that has gained access through the portal web server 150 (FIG. 1). In some embodiments, portal web server access only permits review of data stored in the central computing device 100. In such embodiments, no data may be supplied to the central computing device 100 through the portal web server 150. A requester with access through the portal web server 150 may be able to generate reports regarding the non-PHI data and do data searches by anonymous key, such as the clinical identification code. In alternate embodiments, a requester using the portal web server 150 may be able to modify data previously submitted or as specifically permitted by an administrator.

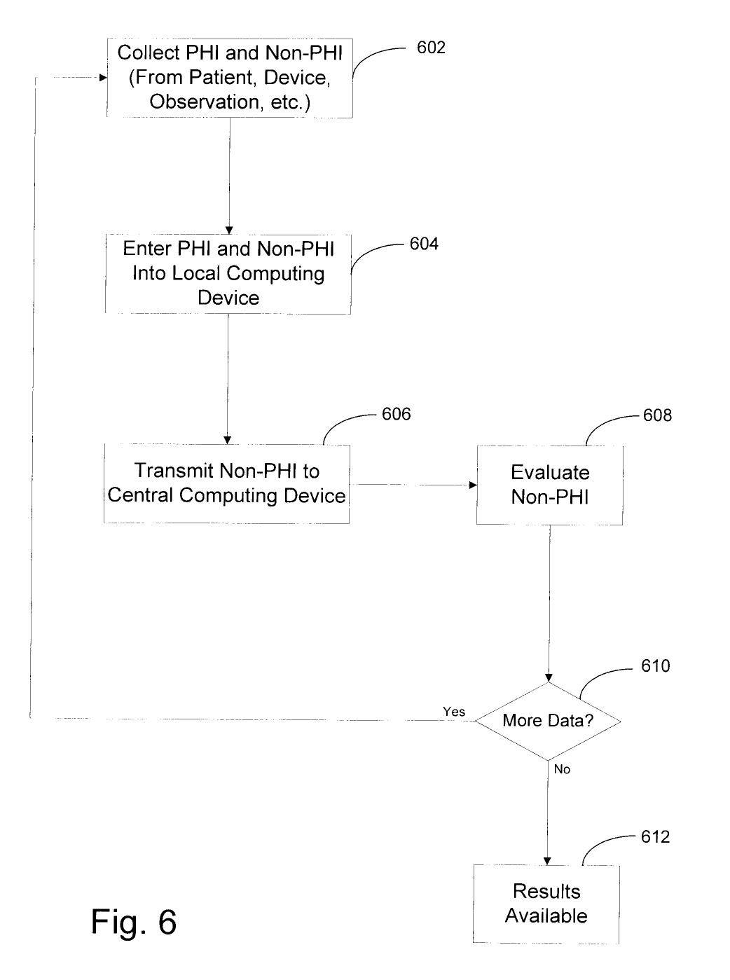

A method embodiment of the invention is represented in FIG. 6. The method may be undertaken to evaluate medical outcomes resulting from implantation of a medical device. As illustrated, the first act of the method is to collect protected health information (PHI) and non-PHI from a patient in which the medical device has or will be implanted (step 602). Examples of the types of information that may be collected include, but are not limited to: name, address, contact information, date of birth, Social Security number Medicare number, sex, marital status, race, educational level, work status, alcohol use, tobacco use, illness and disease, surgical history, prescription drug use, medical and drug payer, general physical condition, mental condition, pain self-assessment, activity self-assessment, physical assessment, including descriptions of symptoms, motor function, sensory function, reflexes, ranges of motion, Waddell Signs, radiographic, surgery data, adverse events, discharge status, post operative status, and dates of appointments. Collection of information may occur in writing, through a computer interface, verbally with transcription or voice recognition, or by any other effective method. The information may also be passed from one computing device to another with storage of the information in the one or more computers' memory components. Communication may be automatic or may be in response to user commands.

Another act of the method represented in FIG. 6 includes entering at least a portion of the PHI and the non-PHI into a local computing device 1 (FIGS. 1, 2) (step 604). The local computing device 1 may be one of the one or more computers specified above, and may be the last computer to which the information is passed. Information may be directly entered into the local computing device 1 in some embodiments.

As illustrated in FIG. 6, some or all of at least a portion of the non-PHI is transmitted to a central computing device 100 in some embodiments (step 606). Transmitted portions of the non-PHI may also be associated with an identifier, wherein in the local computing device 1 (FIGS. 1, 2), the identifier is associated with portions of the PHI.

The transmission of PHI to the central computing device 100 is prevented in some embodiments. The prevention of transmission may be driven from either the local computing device 1 or the central computing device 100 side of the system. The local computing device 1 may prevent transmission by not allowing PHI data to be available for transmission. Alternatively, or in addition, the central computing device 100 may prevent transmission of PHI by not being configured to receive PHI, by rejecting receipt of PHI, or by any other effective means.

FIG. 6 includes an act of evaluating at least portions of the non-PHI transmitted to the central computing device 100 (FIGS. 1, 2) (step 608). The act of evaluating the information may include tracking device performance, correlating any of the large number of recorded patient characteristics with device performance, identifying the need for additional or follow-up information, or any other act of evaluation that be useful in determining the success or failure of a device, method, or treatment. The non-PHI may be evaluated as identified by one or more identifiers such as the clinical identification codes.

In some circumstances, additional data may be useful in evaluating the performance of a medical device after an initial evaluation has been accomplished. FIG. 6 illustrates a decision step entitled “More Data?” wherein some embodiments of the invention provide an opportunity for additional data to be collected (step 610). This decision step may be presented as a result of a passage of a specified period or time, may result from a user-initiated request, may result from a particular algorithm that requires multiple data entries, or may be initiated for any reason that promotes the evaluation of a medical device. If more data is requested, the method returns to the collection of PHI and non-PHI act in some embodiments. Collection of PHI and non-PHI may include the act of collecting information two or more times. Repeated collections of data may be useful to chronicle performance of the implant. If more data is not requested, the embodiment of the invention illustrated in FIG. 6 then makes results of the data collections and evaluations available (step 612). In some embodiments, the results of the data collections and evaluations may be available for viewing before the final step is reached, or may be available while a request for a response to the question of more data remains open.

In some embodiments, non-PHI stored on the central computing device 100 may be accessed from a computing device other than the local computing device 1. For example, a computer may access the non-PHI stored on the central computing device 100 through the portal web server 150.

Embodiments of the invention may include a computer readable media containing instructions to enable collection of clinical information. The computer readable media may be a compact disc, digital versatile disc, hard disc, computer or similar device with pre-loaded software, non-volatile memory device, memory card, memory stick, floppy disc, or any other media capable of recording computer instructions. The instructions of some embodiments include instructions to display data entry pages into which protected health information (PHI) and non-PHI may be added; instructions to store PHI and non-PHI in a local database; instructions to communicate non-PHI over a network; and instructions restricting communication of PHI over the network. The computer instructions may be executable on a single computer system or on a number of computers that are configured to execute part or all of the instructions cooperatively.

While embodiments of the invention have been illustrated and described in detail in the disclosure, the disclosure is to be considered as illustrative and not restrictive in character. All changes and modifications that come within the spirit of the invention are to be considered within the scope of the disclosure.

What is claimed is:

1. A computer system for collecting clinical information regarding degrees of success or failure resulting from implantation of a medical device in a patient comprising:
a local computing device on which protected health information (PHI) and non-PHI are stored, the local computing device including at least:
an authentication sequence wherein access to functionality of the local computing device is controlled,
a tasking sequence wherein interfaces for inputting and reading PHI and non-PHI are presented, and
a communications interface capable of communicating non-PHI over a network, but restricted from communicating PHI over the network; and
a central computing device for receiving non-PHI from the local computing device and for processing non-PHI;
wherein non-PHI is correlated with an identifier, and the identifier is associated with portions of PHI in the local computing device.

2. The computer system of claim 1 wherein the local computing device includes a Universal Serial Bus (USB) memory device.

3. The computer system of claim 1 wherein the local computing device includes a computer with at least a processor, a memory device, and a bus, and wherein the bus is for communicating information at least between the processor and the memory device.

4. The computer system of claim 1 wherein the local computing device includes a biometric scanner for use in the authentication sequence.

5. The computer system of claim 1 wherein the tasking sequence includes an initialization sequence wherein the status of the authenticated local computing device is evaluated.

6. The computer system of claim 1 wherein the tasking sequence includes an initialization sequence wherein software code stored in the local computing device is compared with software code stored in the central computing device.

7. The computer system of claim 6 wherein if the software code stored in the local computing device is an earlier version than the software code stored in the central computing device, the local computing device software code is updated.

8. The computer system of claim 1 wherein the tasking sequence includes code to launch container software to enable the local computing device to fetch, decrypt, and modify locally stored PHI.

9. The computer system of claim 1 wherein the local computing device includes a local identifier.

10. The computer system of claim 1 wherein computer code enabling the interfaces for inputting and reading PHI and non-PHI is stored on the local computing device in hypertext markup language (HTML).

11. The computer system of claim 1 wherein the local computing device includes a planning module that uses PHI and non-PHI to calculate future patient compliance actions.

12. The computer system of claim 1 wherein the central computing device includes a maintenance module to perform maintenance on the local computing device.

13. The computer system of claim 12 wherein the maintenance module performs maintenance on the local computing device in response to commands issued from the local computing device.

14. The computer system of claim 1 further comprising a portal through which non-PHI may be accessed by a computing device other than the local computing device.

15. The computer system of claim 1 further comprising a data storage device connectable to the local computing device for storage of backup data.

16. A computer system for collecting clinical information comprising:
a local computing device comprising:
data entry pages, and
a local database capable of receiving data from the data entry pages,
wherein protected health information (PHI) and non-PHI are stored in the local database, and
wherein the local computing device is capable of communicating over a network, but restricted from communicating PHI over the network; and
a central computing device for receiving non-PHI from the local computing device and for processing the non-PHI comprising:
a web server connectable with the local computing device for receiving information over the network, and
a database server for storing and processing non-PHI.

17. A clinical evaluation system comprising:
a medical device for treating a medical condition;
a local computing device into which information is input, the information comprising:
protected health information (PHI),
non-PHI, and
medical implant performance information related to treatment of the medical condition,
wherein information regarding the performance of the medical implant may include one or both of PHI and non-PHI; and
a central computing device connectable to the local computing device through a network;
wherein the central computing device is enabled to receive non-PHI, but not able to receive PHI from the local computing device.

18. The clinical evaluation system of claim 17 wherein the medical device is a spinal arthroplasty device.

19. A local computing device comprising:
a memory device in which protected health information (PHI) and non-PHI are stored; and
computer readable instructions providing a communications interface that enables the local computing device to transmit non-PHI over a network to another computing device, but restricts the local computing device from communicating PHI over the network;
wherein the local computing device is a portable device retained within the control of a health care provider.

20. A method of evaluating medical outcomes resulting from implantation of a medical device comprising:
collecting protected health information (PHI) and non-PHI from a patient in which the medical device has or will be implanted;
entering at least a portion of the PHI and the non-PHI into a local computing device;
transmitting at least a portion of the non-PHI to a central computing device;
preventing transmission of the PHI to the central computing device; and
evaluating at least portions of the non-PHI transmitted to the central computing device.

21. The method of claim 20 further comprising associating transmitted portions of the non-PHI with an identifier, wherein in the local computing device the identifier is associated with portions of PHI.

22. The method of claim 21 wherein evaluating at least portions of the non-PHI includes evaluating the non-PHI in association with one or more identifiers.

23. The method of claim 20 wherein collecting PHI and non-PHI includes collecting information two or more times with regard to a patient to chronicle performance of the implant.

24. The method of claim 20 further comprising accessing non-PHI stored on the central computing device from a computing device other than the local computing device.
